SEQUENCE LISTING (22 sequences)

SEQ ID NO: 1 - DNA, 62 bp, Artificial Sequence
  Synthetic oligonucleotide
tctagaataacttcgtataatgtatgctatacgaagttatgactggccaaactgatctcaag

SEQ ID NO: 2 - DNA, 56 bp, Artificial Sequence
  Synthetic oligonucleotide
ataacttcgtatagcatacattatacgaagttatatggtgtctgttttctacgtgt

SEQ ID NO: 3 - DNA, 40 bp, Artificial Sequence
  Synthetic oligonucleotide
agggcgaattgggcccgacgtcagcacgttcgatctagca

SEQ ID NO: 4 - DNA, 40 bp, Artificial Sequence
  Synthetic oligonucleotide
ccatgcttagttacaatgcttagccgatctaaaagtggag

SEQ ID NO: 5 - DNA, 40 bp, Artificial Sequence
  Synthetic oligonucleotide
tcttcgtcatcccacctagcgattgcttaccattgtatgc

SEQ ID NO: 6 - DNA, 40 bp, Artificial Sequence
  Synthetic oligonucleotide
ggagctctcccatatggtcgactccatgtcacagtttcgc

SEQ ID NO: 7 - DNA, 20 bp, Artificial Sequence
  Synthetic oligonucleotide
caagggcatcaaactagctg

SEQ ID NO: 8 - DNA, 20 bp, Artificial Sequence
  Synthetic oligonucleotide
gtttaacacagccagatggg

SEQ ID NO: 9 - DNA, 40 bp, Artificial Sequence
  Synthetic oligonucleotide
agggcgaattgggcccgacgtcctattgttcgattcggcg

SEQ ID NO: 10 - DNA, 40 bp, Artificial Sequence
  Synthetic oligonucleotide
ccatgcttagttacaatgcttatgtgcaggtacaacggaa

SEQ ID NO: 11 - DNA, 40 bp, Artificial Sequence
  Synthetic oligonucleotide
gcatacaatggtaagcaatcgcaagctctaagcgcgatgt

SEQ ID NO: 12 - DNA, 40 bp, Artificial Sequence
  Synthetic oligonucleotide
ggagctctcccatatggtcgactgattctgtcgcccaact

SEQ ID NO: 13 - DNA, 20 bp, Artificial Sequence
  Synthetic oligonucleotide
ccatatgattctgtgcctgc

SEQ ID NO: 14 - DNA, 20 bp, Artificial Sequence
  Synthetic oligonucleotide
cgaggagtatccttccacca

SEQ ID NO: 15 - DNA, 20 bp, Artificial Sequence
  Synthetic oligonucleotide
tcctggaggcagaagaactt

SEQ ID NO: 16 - DNA, 20 bp, Artificial Sequence
  Synthetic oligonucleotide
agcccttctgactcacgtat

SEQ ID NO: 17 - DNA, 962 bp, Artificial Sequence
  Synthetic DNA
atggaggtcggcttcctcggactgggtattatgggcaaggctatgtcgatgaacctgctcaagaacggctttaaggtcactgtgtggaaccgaaccctgtctaagtgtgacgagctggtggagcacggcgcttctgtctgtgagtcccccgccgaggtcatcaagaagtgcaagtacaccattgccatgctgtctgacccctgtgccgctctgtccgtggtcttcgacaagggcggagtgctggagcagatctgcgagggcaagggatacattgacatgtctaccgtcgacgctgagacttctctgaagatcaacgaggccattaccggaaagggtggccgattcgtggagggtcccgtctctggatccaagaagcccgccgaggacggtcagctgatcattctggccgctggagacaaggctctgttcgaggagtctatccccgccttcgacgtgctgggaaagcgatccttctacctgggtcaagtgggtaacggcgccaagatgaagctgatcgtcaacatgattatgggatctatgatgaacgctttctccgagggtctggtcctggccgacaagtctggcctgtcttccgacaccctgctggacattctggacctgggtgctatgaccaaccccatgttcaagggaaagggtccctccatgaccaagtcttcctaccctcccgccttccccctgaagcaccagcagaaggacatgcgactggccctggctctgggcgacgagaacgctgtgtccatgcctgtggccgctgccgctaacgaggccttcaagaaggctcgatctctgggcctgggcgacctggacttctccgccgtgatcgaggctgtcaagttcgccgagtacatcgacggaattctgcgaatggtcaccggaggtattacttctaccgccgctatgggtgctggtgtgaccgaggatcagttcaagtctaagctgta

SEQ ID NO: 18 - protein, 18 aa, Yarrowia lipolytica
MLAFKSLRPSAVSRLATS

SEQ ID NO: 19 - protein, 34 aa, Yarrowia lipolytica
MLRHALLKRTPVLPRLTSRKAFVPLIQKRKYSDD

SEQ ID NO: 20 - DNA, 34 bp, Artificial Sequence
  Synthetic oligonucleotide
ggcgcactgcagatgaaaacccgtacacaacaaa

SEQ ID NO: 21 - DNA, 36 bp, Artificial Sequence
  Synthetic oligonucleotide
gcaattcccgggttagaactgcgattcttcagtgga

SEQ ID NO: 22 - DNA, 999 bp, Artificial Sequence
  Synthetic DNA
atgacccgaacccccgtgaacgtcaccgtcaccggcgctgctggtcaaatcggctacgccttattattcagaatcgcctccggtcagctgctgggtgctgacgtccccgtcaagctgcgactgctgggttctacccgatctttaaaggctgctgagggcaccgccatggagctggatgactgcgccttccctttattacaaggtatcgagatcactgatgaccccaacgtggccttcgacggcgctaacgtggctttattagtgggcgctcgacctcgaaccaagggtatggagcgaggcgacctcctcgaggccaacggtggcatctttaagccccaaggtaaggccatcaacgaccacgccgccgacgacatcaaggttttagtcgtgggcaaccccgccaacaccaacgctttaatcgctcaagctgccgctcccgacgtgcccgccgaacgattcactgccatgacccgactcgaccacaaccgagctttaacccagctcgccaagaagaccggttccactgtggccgacattaagcgactgaccatctggggcaaccactccgccacccagtaccccgacatctttcacgccaccgtcgccggcaaaaacgccgccgagaccgtcaacgacgagaagtggctggctgacgagtttatccccactgtcgccaagcgaggcgccgctatcatcgaggctcgaggcgcttcttccgctgcttctgccgccaacgccgccatcgaccacgtctacacttgggtgaatggcaccgccgagggcgactggacctctatgggtatcccctccgacggctcctacggtgtgcccgagggtatcatctcctccttccccgtcaccaccaaggacggttcctacgagattgtccaaggtctggacatcaacgagttctcccgagcccgaatcgacgcctctgtgaaggagctgtccgaggagcgagaagccgtccgaggtctgggtctgatctctaagctgtaa

(19) United States
(12) Patent Application Publication
(10) Pub. No.: US 2022/0127648 A1
(43) Pub. Date: Apr. 28, 2022

(54) GENETICALLY ENGINEERED YEAST YARROWIA LIPOLYTICA AND METHODS FOR PRODUCING BIO-BASED GLYCOLIC ACID

(71) Applicant: INTEGRATED LIPID BIOFUELS LLC, Airway Heights, WA (US)

(72) Inventors: Xiaochao XIONG, Pullman, WA (US); Shulin CHEN, Pullman, WA (US)

(21) Appl. No.: 17/425,288

(22) PCT Filed: Jan. 23, 2020
(86) PCT No.: PCT/US2020/014855

§ 371 Date: Jul. 22, 2021

Related U.S. Application Data
(60) Provisional application No. 62/795,927, filed on Jan. 23, 2019.

(51) Int. Cl. C12P 7/42; C12N 15/81; C12N 15/52; C12N 1/16; C12N 9/04

(52) U.S. Cl. CPC C12P 7/42; C12N 15/815; C12N 15/52; C12N 1/16; C12N 2810/85; C12Y 101/01026; C12Y 101/0104; C12N 2800/102; C12N 9/0006

(57) ABSTRACT

The present disclosure provides a method for genetically engineering Yarrowia lipolytica host cell for producing glycolic acid from organic wastes. A subject genetically engineered Y. lipolytica cell comprises the disrupted native genes encoding malate synthase, heterologous enzyme of glyoxylate reductase targeted in the different cellular compartments including mitochondria, peroxisome and cytosol, and a mutant NADP⁺-dependent malate dehydrogenase. The pathway with a theoretical yield as high as that 1 g of acetic acid can be converted to 1.27 g of glycolic acid without carbon loss was engineered for glycolic acid production. The methods particularly include process for production of volatile fatty acids (VFAs) mainly comprised of acetic acid from organic waste, and then use of resultant VFAs for biosynthesis of glycolic acid by recombinant Y. lipolytica.

### CROSS-REFERENCE TO RELATED APPLICATIONS

The application claims priority as a continuation application to U.S. Provisional Patent Application No. 62/795,927 filed Jan. 23, 2019.

### STATEMENT OF GOVERNMENTAL INTEREST

This invention was made with government support under Grant No. DE-SC00184751 awarded by the U.S. Department of Energy. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480390_401WO_SEQUENCE_LISTING.txt. The text file is 7.0 KB, was created on Jan. 23, 2020, and is being submitted electronically via EFS-Web,

### BACKGROUND

### Technical Field

The present disclosure is in the field of sustainable production of bio-based glycolic acid by using renewable feedstock including organic wastes.

### Description of the Related Art

Glycolic acid (HOCH₂COOH), also known as hydroxyacetic acid and ethanolic acid, is one of the smallest organic molecules with both acid and alcohol functionality. Its unique set of properties makes it ideal for a broad range of applications. As a biodegradable, non-toxic, non-volatile, and phosphate-free chemical, glycolic acid can be used as an efficient cleaning agent with many added benefits such as negligible odor, high solubility in water, and easy rinse. Glycolic acid can also be used as a building block for production of many other chemicals, such as biopolymers poly(glycolic acid) (PGA) and poly(lactic-co-glycolic acid) (PLGA) either by chemical synthesis or biosynthesis. Moreover, glycolic acid is increasingly being used in anti-ageing products and cosmetics developed specially for sensitive skin.

Despite its vital commercial roles and wide applications, glycolic acid occurs naturally only as a trace component in some plants. Different methods have been explored for chemical synthesis of glycolic acid, including carbonylation of formaldehyde with synthesis gas, hydrogenation of oxalic acid, and hydrolysis of the cyanohydrin derived from formaldehyde. These methods involve in use of toxic materials such as formaldehyde and hydrogen cyanide (HCN) for preparation of cyanohydrin, operation under harsh condition such as hydrogenation, and formation of undesirable by-products. There are great opportunities for developing a new, reliable, scalable and safe pipeline for production of glycolate used in as both commodity chemical and specialty chemical required in personal care products.

Thereafter, biosynthesis of glycolic acid by fermentation has been explored as an alternative route to overcome the limitations and disadvantages of the chemical processes. Escherichia coli has been intensively genetically engineered for production of glycolic acid (Deng, Ma et al. 2018). Patents have been filed on genetic engineering of E. coli for glycolic acid production from glucose (WO/2007/141316, WO 2010/108909) and xylose (US 2017/0121717 A1). Other bacterial strains such as Corynebacterium glutamicum were also genetically engineered for production of glycolic acid from sugars, The bacteria are susceptible to phage, resulting in potential infection risk during fermentation process. Additionally, because E. coil is not tolerant to low pH, much base is required to neutralize the fermentation broth.

Thus, a patent (WO 2013/050659 A1) described genetic engineering of eukaryotic cells including yeasts Saccharomyces cerevisiae, Candida krusei and Kluyveromuces lactis, and a filamentous fungus, Aspergillus niger for the production of glycolic acid from glucose. The titer of glycolic acid produced by the recombinant S. cerevisiae was very low, and only reached 0.45 g/L after five-day culture using the mixture carbon source containing 20 g/L glucose and 20 g/L of ethanol. Eukaryotic cells are more challenging for genetic manipulation than bacteria as such manipulation is often hampered by the lack of well-developed genetic tools such as expression vectors. Additionally, the eukaryotic cells such as S. cerevisiae have different organelles such as mitochondria and peroxisomes to isolate and regulate the cellular biochemical reactions. The cellular compartmentalization represents an additional challenge to engineer a productive eukaryotic cell factory for glycolic acid production.

In using sugars as substrates, the maximum theoretical yields are significant lower than 1 g product/g substrate: use of glucose (0.84 g/g) and xylose (0.84 gig through glyoxylate shunt, 0,51 g/g through D-xylulose-1-phosphate) (Salusjärvi, Havukainen et al. 2019). Furthermore, readily supply of low-cost and sustainable carbon source such as cellulosic sugar is still a major challenge as demonstrated by the lack of progress in cellulosic ethanol industry. On the other hand, a significant amount of carbon and energy contained in organic waste streams remains untapped. The U.S. has potentially annual excess of 77 million thy tons of wet waste resource that contains 1.079 quadrillion British thermal units (Btu) of energy. Converting waste to high-value products is a goal the realization of which has long been sought by the engineering community and industry. However, only limited commercial success has been achieved. There are few practical waste utilization technologies available at the commercial level other than anaerobic digestion (AD), but AD alone can only produce biogas instead of diverse, more valuable products such as glycolic acid.

Production of bio-based glycolic acid by genetic engineering of microorganisms from renewable feedstock such as cellulosic sugars is a clear advancement over the petroleum-based chemical, but nevertheless all the existing processes heretofore known suffer from a number of disadvantages and limitations:

(a) The genetically engineered microbial hosts for producing glycolic acid were limited to the model organisms including E coli and S. cerevisiae, and other several microorganisms. None of the strains could produce glycolic acid at both low and high pH, impeding the industrial applications. Lack of the genetic tools for genetic manipulation of non-model host organisms and complicated native cellular metabolism hinder genetic engineering progress.

(b) Although eukaryotic cells such as yeast and fungi contain specialized compartments called organelles, the enzyme for biosynthesis of glycolic acid has been only expressed in the cytosol of the eukaryotic cells. However, pathway compartmentalization has not been employed as a strategy to design and engineer the cell factories for glycolic acid production. Furthermore, the expression systems for targeting the enzymes to a specific organelle such as mitochondria have not been established in some promising organisms.

(c) The theoretical yields for production of glycolic acid from sugars including both glucose and xylose are much lower than 1 g product/g substrate. The low yield generally indicates the low carbon utilization efficiency for producing the target product from substrate. There is a gap in finding an alternative substrate and engineer a more productive pathway for glycolic acid production at a higher theoretical yield.

(d) Currently the processes for production of bio-based glycolic acid rely on the supply of sugars and glycerol. High production cost caused partially by the use of glucose or glycerol as the feedstock prevents the wider acceptance of a bio-based product.

(e) Organic waste can be potentially used as the feedstock. The cost of such a feedstock is negative as it is possible to receive a tipping fee for processing the waste material. This gives a great cost advantage to the technology over the existing technologies. However, the route for converting these negative or low-value wastes to glycolic acid as a high value bioproduct has not been built.

### BRIEF SUMMARY

The present invention provides a method for genetically engineering yeast host cell, Yarrowia lipolytica to be capable of producing glycolic acid. The production strain is not a naturally occurring strain.

The present disclosure provides for a pathway, whose theoretical yield is as high as that 1 g of acetic acid can be converted to 1.27 g of glycolic acid without carbon loss, for glycolic acid production.

In some embodiments of the disclosure, a subject genetically engineered Y. lipolytica comprises the disrupted native genes encoding mal ate synthase, heterologous enzyme of glyoxylate reductase targeted in the different cellular compartments including mitochondria, peroxisome and cytosol, and a mutant NADP⁺-dependent malate dehydrogenase.

The present disclosure provides for the methods for the production of glycolic acid in a subject genetically engineered yeast host at both low and high pH.

The present disclosure provides for the methods for production of volatile fatty acid (VFA) mainly consisting of acetic acid from organic waste, and then use of resultant VFA for biosynthesis of glycolic acid by recombinant Y. lipolytica.

Additionally, the present disclosure provides a system for biosynthesis of glycolic acid, comprising at least one expression cassette comprising a polynucleotide encoding a glycolic acid biosynthesis enzyme operably linked to an expression control sequence. In some embodiments, the glycolic acid biosynthesis enzyme is selected from glyoxylate reductase and NADP⁺-.dependent malate dehydrogenase.

In some embodiments, the system comprises a first expression cassette comprising a polynucleotide encoding glyoxylate reductase operably linked to an expression control sequence and a second expression cassette comprising a polynucleotide encoding NADP⁺-dependent malate dehydrogenase operably linked to an expression control sequence. The glyoxylate reductase may be Glyoxylate Reductase 1 (GLYR1), such as Arabidopsis thaliana GLRY1 (e.g., SEQ ID NO: 17). The NADP⁺-dependent malate dehydrogenase may be from S. coelicolor (e.g., SEQ ID NO: 22).

In some embodiments, the glycolic acid biosynthesis enzyme (e.g., GLYR1.) may include an organelle targeting signal, such as a mitochondria targeting signal or a peroxisome targeting signal.

In some embodiments, the mitochondrial signal is a leading sequence from COX4 (YALI0F03567 g) or a leading sequence from OGDC1 (YALI0E33517 g). The mitochondrial targeting signal may be, for example, at the C-terminus of the glycolic acid biosynthesis enzyme (e.g., GLYR1). In some embodiments, the mitochondrial targeting signal comprises SEQ NO: 19,

In some embodiments, the peroxisome targeting signal is a 33-amino acid peroxisome targeting signal from isocitrate lyase (ICL1). The peroxisome targeting signal may be, for example, at the N-terminus of the glycolic acid biosynthesis enzyme.

In some embodiments, the gene expression cassette(s) of the system includes a heterologous expression control sequence. The expression control sequence(s) may include, for example, a promoter that is functional in a yeast cell (e.g., tef), and/or a terminator that is functional in a yeast cell (e.g., xpr2).

In some embodiments, the system further includes an additional gene expression cassette. For example, the system may include an isocitrate lyase enzyme operably linked to an expression control sequence. As another example, the system may include a citrate synthase operably linked to an expression control sequence.

In some embodiments, the system further includes a gene deletion cassette for deletion of a malate synthase gene. In some embodiments, the system includes a gene deletion cassette for deletion of malate synthase 1 (ms1) and a gene deletion cassette for deletion malate synthase 2 (ms2).

In some embodiments, the gene expression cassette(s) of the systems disclosed herein are present in a yeast transformation vector. The yeast transformation vector may include, for example, a selectable marker, such as leu2 .

Additionally, the present disclosure provides a recombinant yeast cell comprising a knockout of at least one malate synthase gene. In some embodiments, the at least one malate synthase gene is selected from malate synthase 1 (ms1) and malate synthase 2 (ms2). In some embodiments, the yeast cell comprises Y. lipolytica.

In some embodiments, the recombinant yeast cell further comprises at least one polynucleotide encoding a heterologous glycolic acid biosynthesis gene selected from glyoxylate reductase and NADP+-dependent malate dehydrogenase. In some embodiments, recombinant yeast cell further comprises a polynucleotide encoding a heterologous glyoxylate reductase and a polynucleotide encoding a heterologous NADP+-dependent malate dehydrogenase.

Additionally, the present disclosure provides a recombinant yeast cell transformed with any of the systems disclosed herein.

In some embodiments, a recombinant yeast cell as disclosed herein produces an increased level of glycolic acid, relative to a control yeast cell. In some embodiments the recombinant yeast cell converts VFAs into glycolic acid at an increased level, relative to a control yeast cell. In some embodiments, the recombinant yeast cell converts acetic acid into glycolic acid at an increased level, relative to a control yeast cell. In some embodiments, the recombinant yeast cell converts glucose into glycolic acid at an increased level, relative to a control yeast cell. In particular embodiments, the recombinant yeast cell comprises a polynucleotide encoding glyoxylate reductase having an organelle targeting signal selected from a mitochondria targeting signal or a peroxisome targeting signal, and wherein the recombinant yeast cell converts glucose into glycolic acid at an increased level, relative to a recombinant yeast cell transformed encoding a glyoxylate reductase that does not comprise the organelle targeting signal.

The recombinant yeast cell as disclosed herein may be, for example, a dividing cell or a resting cell. In some embodiments, the recombinant yeast cell is immobilized on a support.

Additionally, presented herein is a method of producing a recombinant yeast cell. The method may include introducing into a yeast cell a system of any one of claims 1-30 to produce a recombinant yeast cell; culturing the recombinant yeast cell under conditions sufficient to allow development of a yeast cell culture comprising a plurality of recombinant yeast cells; screening the recombinant yeast cells for expression of a polypeptide encoded by the system; and selecting from the yeast cell culture a recombinant yeast cell that expressed the polypeptide. The screening may be based, for example, on expression of a screenable marker.

Additionally, presented herein is a method of producing glycolic acid, the method comprising culturing a recombinant yeast cell of any one of claims 35-49 under culture conditions sufficient to produce the glycolic acid. The culture conditions may include an amount of a carbon source sufficient to produce the glycolic acid. The carbon source may be, for example, glucose, glycerol, acetic acid, or a combination thereof.

In some embodiments, the culturing results in the production of at least 25 g/L glycolic acid.

The culture conditions may include an amount of glucose sufficient to produce the glycolic acid, and/or an amount of acetic acid sufficient to produce the glycolic acid.

In some embodiments, the culturing results in a maximal theoretical yield of 1.27 g of glycolic acid per 1 g of acetic acid consumed.

In some embodiments, the culture conditions comprise a pH ranging from 1.5 to about 7.0, or about 7.0 to about 10.5. The culture conditions may be, for example, buffered or non-buffered.

Additionally, the present disclosure provides a method of producing volatile fatty acids (UFAs) from organic waste, the method comprising inoculating a culture medium with an anaerobic sludge and culturing the anaerobic sludge with the organic waste under anaerobic culture conditions sufficient to convert the organic waste into VFAs. The culture conditions for producing VFAs from organic waste may include a temperature in the range of 60-80° C. The organic waste may include, for example, biodegradable plastics, food waste, green waste, paper waste, manure, human waste, sewage, and slaughterhouse waste, lignocellulosic biomass, or a combination thereof. In some embodiments, the method of producing VFAs from organic waste results in a concentration of VFAs of at least 30 g/L or at least 40 g/L.

Additionally, the present disclosure provides a method of producing glycolic acid from organic waste, the method comprising: producing NvTAs from organic waste by a method disclosed herein; and converting the VFAs to glycolic acid in a separate bioreactor or flask by culturing a recombinant yeast cell as disclosed herein with the VFAs under culture conditions sufficient to convert the VFAs into glycolic acid.

### BRIEF DESCRIPTION OF THE SEVERAL VIEWS OF THE DRAWINGS

FIG. 1 is a diagrammatic representation of glycolic acid production from either traditional feedstock such as sugars or organic waste.

FIG. 2 is a schematic representation of pathway design for biosynthesis of glycolic acid.

FIG. 3 is a diagrammatic map of plasmid pURA3loxp containing Y. lipolytica ura3 gene flanked by two direct repeats of the 34-bp /oxi) sequences.

FIG. 4 is a diagrammatic map of plasmid pURA3-ms lupdo containing 5′ and 3′ homologous arms of ms1 gene, and ura3 gene flanked by two direct repeats of the 34-bp loxP sequences.

FIG. 5 is a diagrammatic map of expression vectors pYlexp1 with a constitutive promoter Tef, and terminator from Xpr2. The plasmids also contain Y. lipolytica leu2 marker gene, and replication origins for both E. coil and Y. lipolytica.

FIG. 6 is a schematic representation of the procedure to delete a targeted gene by homologous recombination. The ura3 gene integrated in Y. lipolytica genome can be further removed by expression of Cre recombinase with plasmid pYlexp1-cre.

FIG. 7 is a diagrammatic map of plasmid pYlmit1 containing a signal peptide (MTS) from Cox4 gene for expression of enzymes in Y. lipolytica mitochondria.

FIG. 8 shows enhanced green fluorescent protein (EGFP) expressed with the signal peptide from Cox4 gene. The fluoresce generated from EGFP overlapped well with fluorescent emission from the dye that stains mitochondria.

FIG. 9 is a diagrammatic map of plasmid pYlmit1-GLYR1 for expression of GLYR1 encoding glyoxylate reductase from Arabidopsis thaliana in Y. lipolytica mitochondria.

FIG. 10 is a diagrammatic representation of plasmid pYlexp1-GLYR1 for expression of GLYR1 encoding glyoxylate reductase from A. thaliana in Y. lipolytica cytosol.

FIG. 11 is a diagrammatic representation of plasmid pYlpero-GLYR1 for expression of GLYR1 encoding glyoxylate reductase from A. thaliana in Y. lipolytica peroxisome.

FIG. 12 is a diagrammatic representation of cloning procedure to combine the expression cassettes of aceA encoding isocitrate lyase gene and gltA encoding citrate synthase from E. coil to co-express aceA and gltA in Y. lipolytica mitochondria.

FIG. 13 is a diagrammatic representation of cloning procedure to generate plasmid pGlAc-ura3 by replacing DNA fragment containing Y. lipolytica replication site and leu2 marker with ura3 marker,

FIG. 14 shows the growth of parent strain Y. lipolytica Polf and double knockout GLO9 (Δms1Δms2) on 20 g/L glucose.

FIG. 15 shows the growth of parent strain Y. lipolytica Polf and double knockout GLO9 (Δms1Δms2) on 30 g/L acetic acid.

FIG. 16 shows glycolic acid production from 40 g/L glucose by Y. lipolytica GLO10 expressing GLYR1 from A. thaliana in mitochondria, GLO11 expressing GLYR1 in peroxisome, and GLO12 expressing GLYR1 in cytosol.

FIG. 17 shows glycolic acid production from 30 g/L acetic acid by Y. lipolytica recombinants GLO10, GLOI1 and GLO12.

FIG. 18 shows glycolic acid production from 40 g/L glucose by Y. lipolytica recombinants GLO10, GLO15 and GLO16.

FIG. 19 shows glycolic acid production, concentration of glucose, and growth of Y. lipolytica GLO16 culture in presence of 40 g/L glucose.

FIG. 20 shows glycolic acid production from 30 g/L acetic acid by Y. lipolytica recombinants GLO10, GLO15, GLO16 and GLO20.

FIG. 21 shows the time-course curve of acetic acid production from food waste.

FIG. 22 shows glycolic acid production by Y. lipolytica GLO20 from VFA generated from food waste.

### DETAILED DESCRIPTION

In various embodiments, the present disclosure provides systems and methods for biosynthesis of glycolic acid. In particular, the system comprises at least one expression cassette comprising a polynucleotide encoding a glycolic acid biosynthesis enzyme operably linked to an expression control sequence. Also provided are recombinant yeast cells (e.g., transformed with a system disclosed herein).

As used herein, a polynucleotide or polypeptide is “recombinant” when it is artificial or engineered, or derived from an artificial or engineered protein or nucleic acid. For example, a polynucleotide that is inserted into a vector or any other heterologous location, e.g , in a genome of a recombinant organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene is recombinant.

“Variant” protein is intended to mean a protein derived from the protein by deletion truncation at the 5′ and/or 3′ end) and/or a deletion or addition of one or more amino acids at one or more internal sites in the native protein and/or substitution of one or more amino acids at one or more sites in the native protein. Variant proteins encompassed are biologically active, that is they continue to possess the desired biological activity of the native protein.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The term “stably incorporated” in cell or explant refers to the integration of the polynucleotide into the genomic DNA of the cell.

“Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or noncontiguous. When used to refer to the joining of two protein coding regions, by operably linked is intended that the coding regions are in the same reading frame. The cassette may additionally contain at least one additional coding sequence/gene to be co-transformed into the organism. Alternatively, the additional coding sequences/gene(s) can be provided on multiple expression cassettes. Such an expression cassette is provided with a plurality of restriction sites and/or recombination sites for insertion of a coding polynucleotide of interest or active variant or fragment thereof to be under the transcriptional regulation of the regulatory regions (e.g., promoter). The expression cassette may additionally contain selectable marker genes.

“Expression cassette” refers a polynucleotide encoding a polypeptide of interest operably linked to at least one polynucleotide encoding an expression control sequence. The expression cassette can include in the 5′-3′ direction of transcription, a transcriptional and translational initiation region (i.e., a promoter), polynucleotide encoding a polypeptide of interest or active variant or fragment thereof, and a transcriptional and translational termination region (i.e., termination region) functional in yeast. The regulatory regions (i.e., promoters, transcriptional regulatory regions, and translational termination regions) and/or the polynucleotide or active variant or fragment thereof may be native:/analogous to the host cell or to each other. Alternatively, the regulatory regions and/or the polynucleotide of or active variant or fragment thereof may be heterologous to the host cell or to each other.

“Gene deletion cassette” refers a polynucleotide that, when expressed in a host cell, causes deletion of at least a portion of a gene of interest, such that the gene is not expressed. A gene deletion cassette may include a region of homology to a sequence upstream of a gene of interest, followed by a first repeat sequence (e.g., hisG or loxP), followed by a marker (e.g., ura3) followed by a second repeat sequence, followed by a region of homology to a sequence downstream of the gene to be deleted. In some embodiments the gene deletion cassette includes loxP repeat sequences and a ura3 marker.

“Transformation” as used herein refers to the uptake of DNA (e.g., in the form of an expression cassette) into a yeast cell.

“Yeast transformation vector” as used herein refers to a DNA molecule used as a vehicle of delivery foreign genetic material into a yeast cell. An expression cassette may be a component of a vector (e.g., a yeast transformation vector), and multiple expression cassettes may be present together in a single vector. For example, a vector may encode multiple proteins of interest (e.g., two glycolic acid biosynthesis enzymes or a single glycolic acid biosynthesis enzyme and a selectable marker or screenable marker).

“Expression control sequence” refers to a segment of a nucleic acid molecule which is capable of increasing or decreasing the expression of a polypeptide encoded by the expression cassette. Examples of expression control regions include promoters, transcriptional regulatory regions, and translational termination regions.

The termination region may be native with the transcriptional initiation region, may be native with the operably linked polynucleotide or active variant or fragment thereof, may be native with the yeast cell, or may be derived from another source (i.e., foreign or heterologous) to the promoter, the polynucleotide or active fragment or variant thereof, the yeast cell, or any combination thereof. Examples of terminators functional in yeast can be found, for example, in Curran et al., Metab Eng. 2013 September: 19:88-97.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Prov. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94.

Promoters include constitutive and regulated promotes. Examples of promoters functional in yeast can be found, for example, in Peng et al., Microb Cell Fact (2015) 14:91.

A “control” or “control yeast” or “control yeast cell” provides a reference point for measuring changes in phenotype of the subject yeast cell, and may be any suitable yeast cell. A control yeast cell may comprise, for example: (a) a wild-type or native yeast cell, i.e., of the same genotype as the starting material for the genetic alteration which resulted in the subject yeast cell; (b) yeast cell of the same genotype as the starting material but which has been transformed with a null construct (i.e., with a construct which has no known effect on the trait of interest, such as a construct comprising a marker gene); or (c) the subject yeast cell itself, under conditions in which the gene of interest (e.g., the gene encoding a glycolic acid biosynthesis enzyme) is not expressed.

Various methods can be used to introduce a sequence of interest into a yeast cell. “Introducing” is intended to mean presenting to the yeast cell the polynucleotide or polypeptide in such a manner that the sequence gains access to the yeast cell. The methods of disclosed herein do not depend on a particular method for introducing a sequence into yeast, only that the polynucleotide or polypeptides gains access to the yeast cell. Methods for introducing polynucleotide or polypeptides into yeast cells are known in the art including, but not limited to, stable transformation methods, transient transformation methods, and virus or virus-like element-mediated methods.

In the present description, the term “about” means +20% of the indicated range, value, or structure, unless otherwise indicated. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

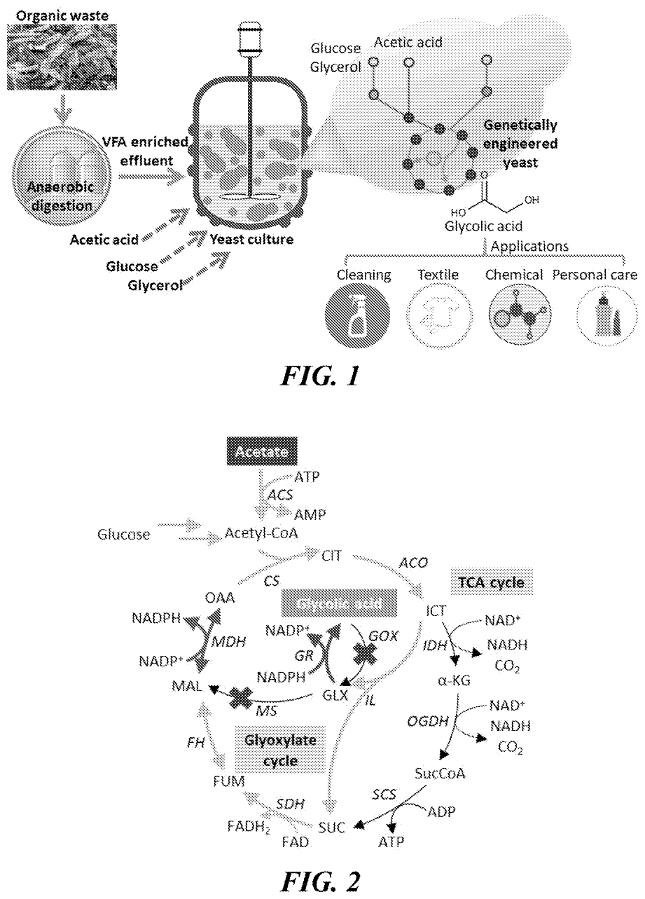

The present disclosure relates to a non-conventional yeast which is genetically engineered to produce glycolic acid. The genetically engineered yeast strain can be used for production of glycolic acid from the common substrates such as glucose and glycerol, a novel substrate acetic acid exerting a toxic effect to other microorganisms, and raw material of organic waste (FIG. 1).

In one embodiment, a non-conventional yeast Y. lipolytica has been genetically engineered for the production of glycolic acid. As a Generally Recognized As Safe (GRAS) organism, Y. lipolytica has been widely used for industrial production of a suite of chemicals such as lipid mainly consisting of triacylglycerol (TAG) and lipid-derived molecules such as eicosapentaenoic acid (EPA) (Markham and Alper 2018). Non-lipid compounds such as lycopene can also be produced by genetic engineering of Y. lipolytica. Another benefit to using yeast is the avoidance of bacteriophage attacks which could impede glycolic acid production at industrial levels.

In one embodiment, the host Y. lipolytica can use acetic acid and other carboxylic adds for the growth and glycolic add production (FIG. 15). Acetic acid and other carboxylic acids including propionic acid and butyric acid are considered inhibitory to most microorganisms including E. coil and S. cerevisiae. Although recombinant E. coli was further employed for production of glycolate from acetate, but it could grow only in acetate with content lower than 5 g/L (Li, Chen et al. 2019). Y. lipolytica on the other hand, can readily convert acetic acid to product and cell biomass, thus making it possible to utilize a variety of substrates that are less efficiently utilized by other host cells.

In one embodiment, the theoretical yield of a pathway is one mole glycolic acid per mole acetic acid as shown in Table 1. In this designed pathway, two heterologous genes encoding glyoxylate reductase (GR) and a mutant NADP⁺-dependent malate dehydrogenase (MDH) from S. coelicolor A3(2) (Ge, Song et al. 2014) need to be introduced into Y. lipolytica for producing glycolic acid from acetic acid through the glyoxylate shunt and TCA cycles (Salusjärvi, Havukainen et al. 2019) (FIG. 2). Acetic acid can be converted to acetyl-CoA through the native acetyl-CoA synthase (ACS2) in Y. lipolytica at a loss of two moles of ATP equivalents, as ATP is transformed into AMP (Eq. 1). Citric acid is formed by the combination of acetyl-CoA and oxaloacetate and is then converted to isocitrate. Isocitrate is cleaved by isocitrate lyase to generate glyoxylate and succinate (Eq. 3), and the former is accumulated when malate synthase is disrupted. Succinate is transformed into fumarate, generating FADH₂ (Eq. 5). By replacing the native NAD⁺-dependent MDH with the enzyme with altered coenzyme specificity (Ge, Song et al. 2014), it can provide NADPH to support glycolic acid production from glyoxylic acid catalyzed by GR (Eq. 6). Oxalate can be combined with acetyl-CoA to start the next run of biosynthesis of glyoxylic acid. Eq. 7 describes generation of ATP, which can be used to activate acetate into acetyl-CoA. Then the stoichiometry of biosynthesis of glycolic acid from acetic acid can be obtained (Eq. 8).

TABLE 1
Calculation of efficiency for production
of glycolic acid from acetic acid

| Equation | Reaction |
|---|---|
| (1) | Acetate + ATP + CoA = Acetyl-CoA + AMP + 2 Pi |
| (2) | Acetyl-CoA + Oxaloacetate + H₂O = Citrate + CoA |
| (3) | Isocitrate = Glyoxylate + Succinate |
| (4) | Glyoxylate + NADPH + H⁺ = Glycolate + NADP⁺ |
| (5) | Succinate + FAD²⁺ = Fumarate + FADH₂ + 2H⁺ |
| (6) | Malate + NADP⁺ = Oxoacetate + NADPH + H⁺ |
| (7) | FADH₂ + O₂ + 2 (H⁺ + ADP + Pi) = 2 ATP + H₂O + FAD²⁺ |
| (8) | Acetate (C₂H₄O₂) + O₂ + 2 H⁺ = Glycolate (C₂H₄O₃) + |
| | H₂O + FAD²⁺ |

As indicated in Table 1, glycolic acid can be produced from acetate with a theoretical yield of 1.27 g/g by the designed pathway. This yield is much higher than the theoretical yields of other carbon sources are used as the substrates for biosynthesis of glycolic acid, such as glucose (0.84 gig) and xylose (0,84 gig through glyoxylate shunt, 0.51 gig through D-xylulose-1-phosphate) (Salusjärvi, Havukainen et al. 2019). The invention overcomes the low yield barrier in glycolic acid production.

The starting strain for genetic engineering was Y. lipolytica Polf (ATCC MYA-2613), which can be obtained from American Type Culture Collection (ATCC). Y. lipolytica Polf is a leucine and uracil-auxotrophic strain, so both leu2 and ura3 from its parent strain, wild-type Y. lipolytica ATCC 20460 can be used as selectable markers for efficient detection and selection of transformants on the selective agar plates lacking leucine and uracil, respectively. To accomplish genetic engineering of the yeast, the chemicals, culture media, kits, plasmids, restriction endonucleases products, and PCR enzymes and reagents are available from the public resources and commercial inventories. The procedures for gene cloning that are now standard in molecular biology (Green and Sambrook 2012), and the specific steps related to genetic engineering of the yeast have been disclosed in embodiment and examples,

In one embodiment, genetic engineering of Y. lipolytica has been carried out for glycolic acid production and further improvement for biosynthesis of target. For genetic engineering of microorganisms especially eukaryotic cells, the considerations include the complexity of native pathways, the existence of organelle organization, and requirement of specific genetic tools such as expression vectors for targeting the enzymes into cellular compartments.

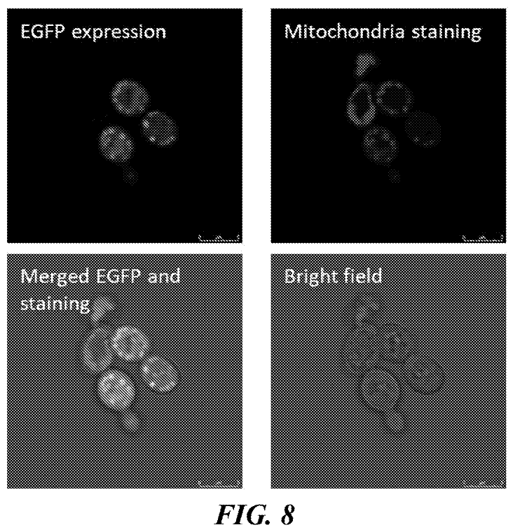

In one embodiment, to express an enzyme in a yeast compartment, a functional signal peptide was used to target the protein to a specific organelle, such as the mitochondrial matrix. N-terminal leading sequences from putative mitochondrial enzymes, cytochrotne c oxidase subunit IV (COX4, YALI0F03567 g) and 2-oxoglutarate dehydrogenase E1 component (OGDC1, YALI0E33517 g) were tested, and their capability to drive the expression of a reporter protein, enhanced green fluorescent protein (EGFP) in yeast mitochondria was verified (FIG. 8). The expression vectors were constructed by use of DNA regions encoding the leading amino acids of the native mitochondria' enzymes to express enzymes in mitochondria.

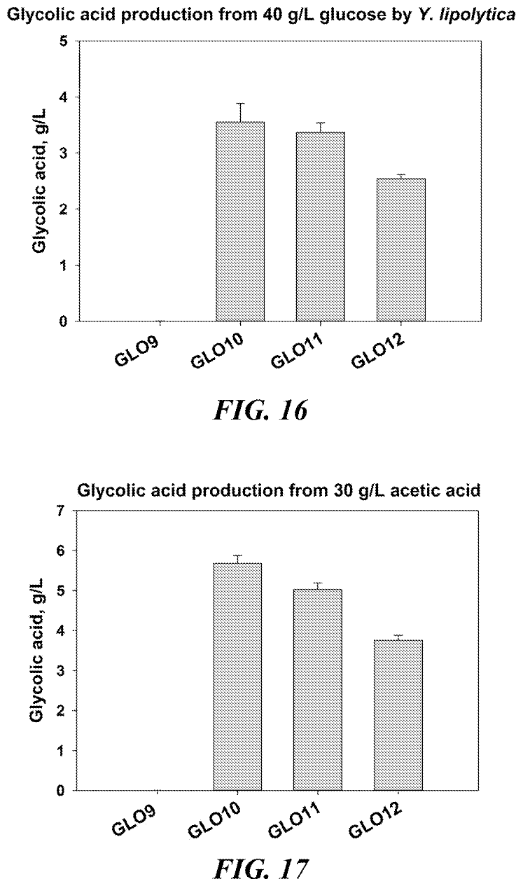

In one embodiment, Y. lipolytica has been genetically engineered by employment of the strategy of pathway compartmentalization. In yeast, the reactions of the glyoxylate shunt and TCA cycle are highly connected, involving in different cellular compartments including cytosol, peroxisomes and the mitochondria. The strains Y. lipolytica expressing gene GLYR1 from A. thaliana encoding glyoxylate reductase 1 were constructed for glycolic acid production, but the expressed enzymes were present in the different cellular organelles including mitochondria, peroxisome and cytosol of these strains. The strain expressing GLYR1 in mitochondria could produce 3.53 g/L of glycolic acid in shaking flask from 40 g/L of glucose in 4 days, which was higher than the contents of glycolic acid produced by the strains expressing the enzyme in peroxisome and cytosol (FIG. 16). Similarly, the strain expressing GLYR1 in mitochondria reached the highest glycolic acid content, 5.68 g/L by using 30 g/L of acetic acid as carbon source (FIG. 17). This result highlights that Y. lipolytica has a great potential for glycolic acid production from acetic acid, and pathway compartmentalization has the specific benefits for design and engineering of this yeast cell factory.

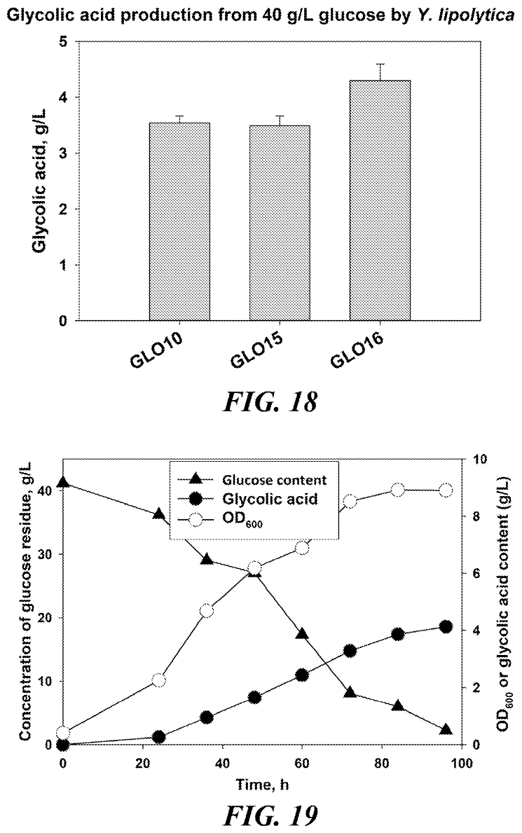

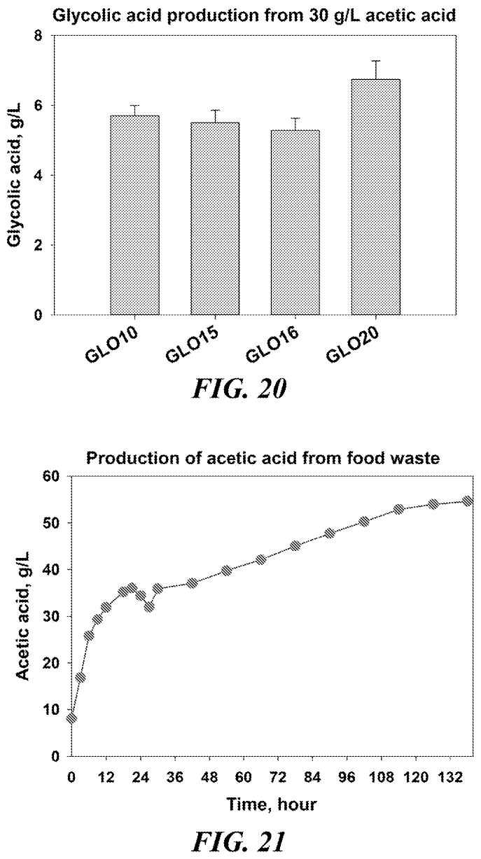

In one embodiment, additional genes have been expressed to further improve glycolic acid production by Y. lipolytica. Co-expression of the genes aceA encoding isocitrate lyase and OA encoding citrate synthase from E. coil in Y. lipolytica strain bearing GLYR1 enabled production of glycolic acid at 4.29 g/L after 96 h cultivation on 40 g/L glucose (FIG. 18). However, expression of aceA and gltA did not improve glycolic acid production from acetic acid (FIG. 20). The strain was developed by introducing mutant gene mut-MDH encoding a modified malate dehydrogenase (MDH) from S. coelicolor A3(2). The titer of glycolic acid production reached 6.74 g/L by cultivation at 96 hour with 30 g/L acetic acid, and a yield at 0.22 g glycolic acid/g acetic acid was achieved (FIG. 20). Glycolic acid can be efficiently produced from acetic acid by genetically engineered yeast (FIG. 17, FIG. 20).

In one embodiment, Y. lipolytica is capable of robust growth under stress conditions of both low pH and high pH. For use of glucose as substrate for production of glycolic acid, pH of the fermentation broth decreased from 6.0 to 2.0 due to secretion of organic acids to supernatant by the cells. For use of acetic acid as substrate for production of glycolic acid, pH increased from 7.0 to 9.45 during cultivation mainly due to utilization of acetic acid. Although a buffer solution can be used for fermentation or acid/base can be added to adjust pH, fermentation without pH control can reduce the risk of contamination and further save use of acid/base.

In one embodiment, VFAs were produced from organic wastes such as food waste by a modified AD process. AD is a commonly accepted process for converting organic wastes to bioenergy in the form of biogas (CH₄ and CO₂). The AD process involves a mixed culture of symbiotic bacteria that mediate the degradation of organic matter ultimately to CH₄, CO₂, and mineralized nutrients. A typical AD process of solids wastes involves multiple steps: disintegration of the waste breaks down the initial solid biomass into separate components; hydrolysis converts relatively large organic compounds, lipids, carbohydrates, and proteins to long chain fatty acids, monosaccharides, and amino acids, respectively; acidogenesis converts VFAs other than acetate, such as propionate and butyrate, to acetic acid and hydrogen; methanogenesis, the last and rate-limiting step in AD, uses formic acid, acetic acid, methanol, and hydrogen as energy sources by various methanogens to generate CH₄ and CO₂ (Agler, Wrenn et al. 2011). VFA production can be improved by enhancing the hydrolysis and acidogenesis rates through physical or chemical pretreatments, addition of enzymes, pH control, redox potential and inoculum optimization, In addition, the chemical 2-bromoethanosulfophate is often added to inhibit methanogenesis.

In one embodiment, a novel hyperthermophilic AD operating at 60-80° C. for production of VFAs from waste streams (FIG. 21). Aside from the generally accepted advantages of AD processes (no sterile conditions or expensive enzymes required, mixed microbial communities can handle complex and variable organic waste streams), using hyperthermophilic AD adds unique benefits for producing VFAs. At these temperatures, methane production ceases as methanogens are not thereto-tolerant. Higher temperatures allow more complete digestion of the feedstock, higher VFA yields, and decreased solid retention times.

In one embodiment, the technology for production of glycolic acid from organic waste is developed by integrating two processes: (1) converting complex waste materials into a group of simple molecules, VFAs mainly consisting of acetic acid, through acidogenesis in AD, and (2) converting the resultant VFAs to the target products in a separate bioreactor or flask by a metabolically engineered yeast strain (FIG. 1). The cost of such a feedstock is negative as it is possible to receive a tipping fee for processing the waste material. This gives a great cost advantage to this invention over the existing technologies. The low-cost strategy can potentially overcome the feasibility barrier and make this technology more competitive in the marketplace.

In one embodiment, the novel bio-based glycolic acid technology takes advantage of both the anaerobic microbial consortia's capacity for handling complex waste, and engineered cell factories for biosynthesis of the target molecule. According to the various embodiments disclosed herein, this opportunity is addressed by providing a cost-effective route to convert these negative or low-value wastes to high value bioproduct (FIG. 1). Although production of bio-based glycolic acid is the main focus of the present disclosure, it should be recognized that the similar platform can be used to produce a variety of other important commodity chemicals and bioproducts by constructing different metabolic pathways in the microbial host. Various organic wastes including wheat straw, corn stover, fruit and vegetable waste, food waste and manure have been processed by AD. Therefore, the technology can potentially have much broader impacts in establishing an industry with various value chains.

### EXAMPLES

### Example 1

Deletion of Genes MS1 and MS2 Encoding Malate Synthase in Y. Lipolytica

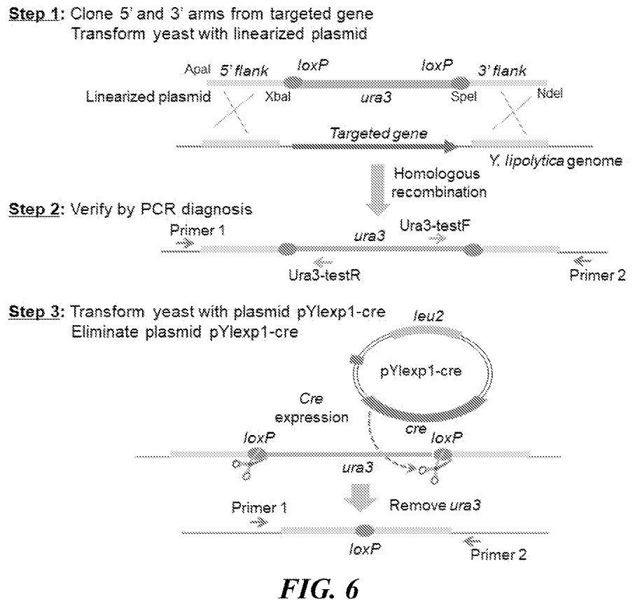

The procedure for deletion of genes in Y. lipolytica has been provided in FIG. 6. The primers and their sequence for deletion of ins1 and ins2 can be found in Table 2. This example provides the detail protocol for deletion of genes in Y. lipolytica.

Step 1: Clone 5′ and 3′ Arms from Targeted Gene and Transform Yeast with Linearized Plasmid

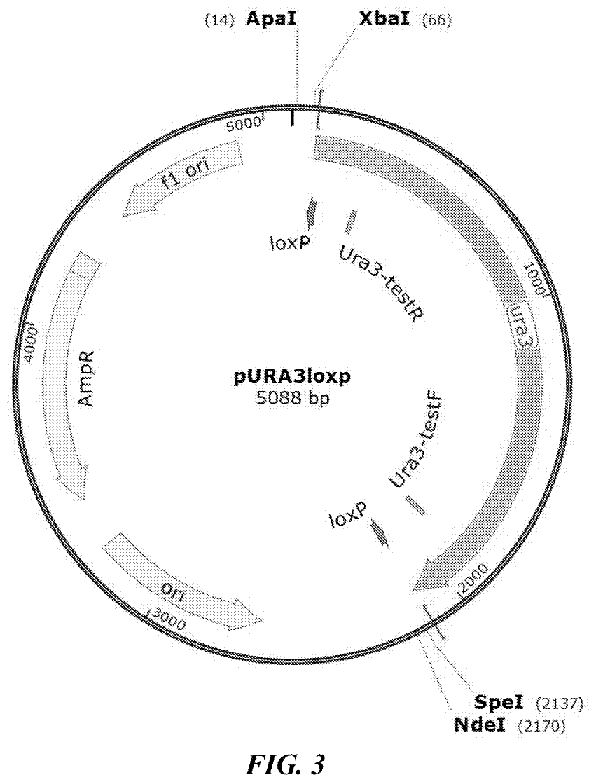

A 2.03-kb DNA fragment of ura3 flanked by loxP sites was obtained by PCR by using primers ura3-F1 (SEQ ID NO 1) and ura3-R1 (SEQ ID NO 2), and genome DNA of Y. lipolytica ATCC 20460 as the template. The PCR product was then cloned into plasmid pGEM-T easy purchased from Promega Corporation according to manufacturer's manual. The resultant plasmid pURA3loxp can be used to generate the vector for disruption of the gene in Y. lipolytica Polf and its derivatives (FIG. 3),

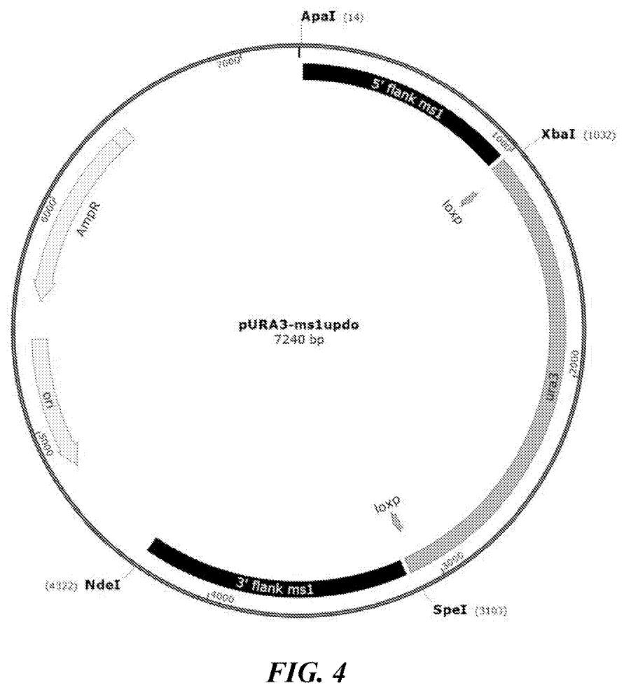

By using genome DNA of Y. lipolytica as the template, the homologous 5′ flank of the targeted gene ms1 with size of 0.97 kb was amplified by PCR with the primers ms1-up1 (SEQ ID NO 3) and ms1-up2 (SEQ ID NO 4), and then inserted into the digested plasmid pURA3loxp after digestion with endonucleases ApaI and XbaI. The resultant plasmid containing the homologous 5′ flank of ns1 was designated pURA3-ms1up. Similarly, 1.17-kb 3′ arm of ms1 was obtained by PCR with primers ms1-do1 (SEQ ID NO 5) and ms1-Do2 (SEQ ID NO 6), and then the digested PCR product was cloned into the sites of SpeI and NdeI in pURA3-ms1up. The resultant plasmid, pURA3-ms1updo contained both 5′ and 3′ arms from ins 1 (FIG. 4). The plasmid pURA3-ms1updo was digested with INdeI. After recovery of the digested product, Y. lipolytica PoIf was transformed with the linearized plasmid pURA3-ms1updo by using the Frozen-EZ Yeast Transformation II Kit (Zymo Research, Irvine, Calif.) based on the manufacturers' guideline. Yeast transformants were grown at 28° C. on the agar plates of selective media, which was composed of 20 g/L of glucose, 6.7 g/L of yeast nitrogen base (YNB w/o amino acids, United States Biological), and 2.0 g/L of complete supplement of amino acids lacking uracil (Drop-out Mix Synthetic Minus Uracil, United States Biological) and 15 g/L agar. After three days, the colonies were visible on the agar plates.

TABLE 2
Primers used for deletion of genes ms1 and ms2

| | | SEQ |
|---|---|---|
| | | ID |
| Primer | Sequence | NO |
| ura3-F1 | TCTAGAATAACTTCGTATAATGTATGCTATAC | 1 |
| | GAAGTTATGACTGGCCAAACTGATCTCAAG | |
| ura3-R1 | ATAACTTCGT ATAGCATACA TTATACGAAG | 2 |
| | TTATATGGTG TCTGTTTTCT ACGTGT | |
| MS1-up1 | AGGGCGAATTGGGCCCGACGTC | 3 |
| | AGCACGTTCGATCTAGCA | |
| MS1-up2 | CCATGCTTAGTTACAATGCTTA | 4 |
| | GCCGATCTAAAAGTGGAG | |
| MS1-Do1 | GCATACAATGGTAAGCAATCGC | 5 |
| | TAGGTGGGATGACGAAGA | |
| MS1-Do2 | GGAGCTCTCCCATATGGTCGAC | 6 |
| | TCCATGTCACAGTTTCGC | |
| MS1-testF | CAAGGGCATCAAACTAGCTG | 7 |
| MS1-testR | GTTTAACACAGCCAGATGGG | 8 |
| MS2-up1 | AGGGCGAATTGGGCCCGACGTC | 9 |
| | CTATTGTTCGATTCGGCG | |
| MS2-up2 | CCATGCTTAG TTACAATGCT TA | 10 |
| | TGTGCAGGTACAACGGAA | |
| MS2-Do1 | GCATACAATGGTAAGCAATCGC | 11 |
| | AAGCTCTAAGCGCGATGT | |
| MS2-Do2 | GGAGCTCTCC CATATGGTCG AC | 12 |
| | TGATTCTGTCGCCCAACT | |
| MS2-testF | CCATATGATTCTGTGCCTGC | 13 |
| MS2-testR | CGAGGAGTATCCTTCCACCA | 14 |
| uar3-testE | TCCTGGAGGCAGAAGAACTT | 15 |
| uar3-testR | AGCCCTTCTG ACTCACGTAT | 16 |

### Step 2: Verify Homologues Recombination by PCR Diagnosis

The single colonies on the selective agar plates were picked up and cultivated in culture tube containing 2 ml of YPD media at 28° C. and a shaking speed of 200 rpm in a shaker. At the same time, the colonies were replicated on YPD plates. The recipe of YPD medium was 10 g/L of yeast extract (Difco), 20 g/L of peptone (Difco), and 20 g/L of glucose, and YPD agar plates were made by adding 15 glL agar (Difco).

After cultivation for two days, the culture was used to extract genomic DNA by using the following protocols. The 1.5 ml cells were harvested by centrifugation at 10,000 g for 5 min, After discarding the supernatant, the cells were suspended in 500 μL of lysis solution containing 200 mM lithium acetate and 1% SDS. The mixture of cells and lysis solution was incubated for 10 minutes at 70° C. to break down the cell wall. The same volume (500 μL) of Phenol: Chloroform: Isoamyl Alcohol (25:24:1, v/v) (Thermo Fisher Scientific) was added into the mixture, and then centrifuged at 13,000 g for 5 minutes after vortex. After centrifugation, 400 ul of aqueous phase (upper phase) was transferred to a new 1.5-ml Eppendorf tube, and two volumes of ethanol (800 ul) were added into the new tube. After mixing, the tubes were kept at −20 DC for 2 hours in a freezer for precipitation of genomic DNA. The samples were centrifuged at 13,000 g for 10 minutes to obtain the genomic DNA. One ml of 70% ethanol was added to the DNA pellet and centrifuged at 13,000 g for 10 minutes to wash DNA. After discarding the washing solution and drying for 10 minutes at room temperature, DNA pellet was dissolved with 50 μL of H₂O or TE buffer (10 mM Tris and 1 mM EDTA, pH 8.0). The extracted genome DNA was used as a template for PCR to verify the deletion of ins/with primer pairs of ms1-testF/uar3-testR (SEQ ID NO 16) and msl-testR/uar3-testF (SEQ ID NO 15) (FIG. 6). Agarose gel electrophoresis of PCR products was carried out to analyze the size and yield. Deletion of ms1 gene in the strain was verified based on the electrophoresis results.

Step 3: Transform Yeast With Plasmid pYlexp1-cre to Remove Marker uar3, and Eliminate Plasmid pYlexp1-cre

The single colony of Y. lipolytica with deleted ms1 gene was cultivated in 20 ml YPD media at 28° C. for 24 hours. The yeast culture was harvested, and transformed with pYlexp1-cre bearing Cre recombinase gene by using the Frozen-EZ Yeast Transformation II Kit (Zymo Research, Irvine, Calif.). Yeast transformants were grown at 28° C. on selective agar plates, which was composed of 20 g/L of glucose, 6.7 g/L of yeast nitrogen base without amino acids, and 2.0 g/L of complete supplement of amino acids lacking leucine (Drop-out Mix Synthetic Minus Leucine, United States Biological) and 15 giL agar. After three days of cultivation at 28° C., the visible colonies were picked up and inoculated into 2-ml YPD media in culture tubes. After culture for 36 hours at 28° C. with a shaking speed at 200 rpm, the cells were plated onto YPD agar plates. The single colonies were then tested for their growth on the selective agar plates lacking either uracil (Drop-out Mix Synthetic Minus Uracil) or leucine (Drop-out Mix Synthetic Minus Leucine) plates. No growth of the strains on both selective agar plates indicates the removal of ura3 marker gene and plasmid pYlexp1-cre curing. The single knockout Δms1 was used for the next round of gene deletion to develop double knockout Δms1Δms2 without ura3 (strain GLO9) by using the same protocol involving step 1-step 3. The strain GLO9 was tested for its growth on glucose and acetic acid, and further engineered by expression of GLYR1 from A. thaliana for glycolic acid production.

### Example 2

### Expression of GLYR1 From A. Thaliana in Y. Lipolytica GLO9 for Glycolic Acid Production

The Y. hpoiytica codon-optimized gene encoding GLYR1 from A. thaliana was synthesized (SEQ ID NO 17). The C E terminal tripeptide, □SRE from GLYR1 was removed during gene synthesis. At the same time, C-terminal 33-amino acid from isocitrate lyase (ICL1, YALI0C16885 g) for peroxisomal localization was fused with GLYR1, and the restriction sites of AAGCTT (for HindIII) and CCCGGG (for SmaI) were introduced into both ends of DNA fragment during synthesis.

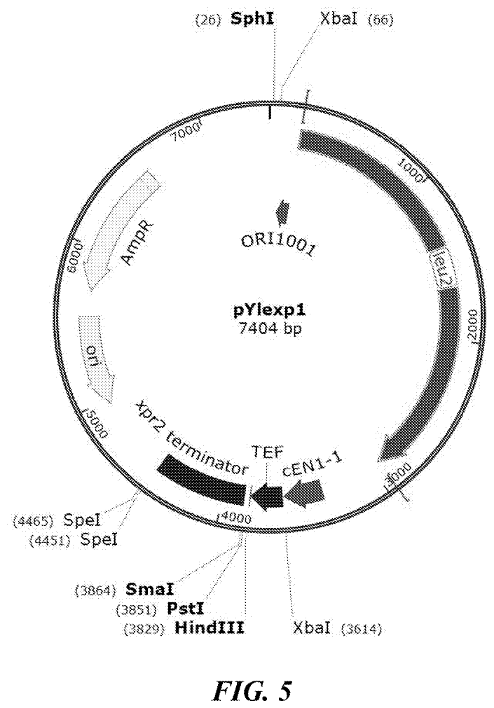

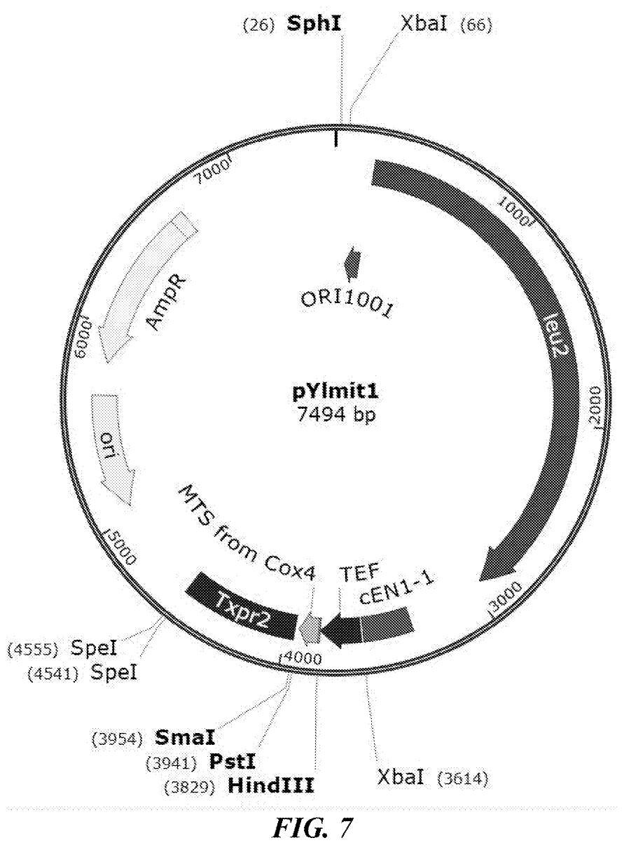

To express gene in Y lipolytica, expression vector pYlexp1 containing a functional 0.20-kb Tef promoter and 0.58-kb xpr2 terminator was constructed (Blazeck, Liu et al, 2011). The plasmid pYlexp1 can replicate in both Y. lipolytica and E. coli because it contains yeast replication origin ORI1001, centromere (CEN) and selection marker leu2 from pS116-Cen1-1(227) (Yamane, Sakai et al. 2008) (FIG. 5), The plasmid pYlexp1 also contains three unique restriction sites for endonucleases HindIII, PstI and SmaI, which can be used to clone and express a gene of interests (FIG. 5). The expression vectors pYlinit1 and pYlmit2 were constructed by use of 18 leading amino acids from COX4 (SEQ ID NO 18) and 34 leading amino acids from OGDC1 (SEQ ID NO 19) encoded DNA regions to express enzymes in mitochondria, respectively (FIG. 7).

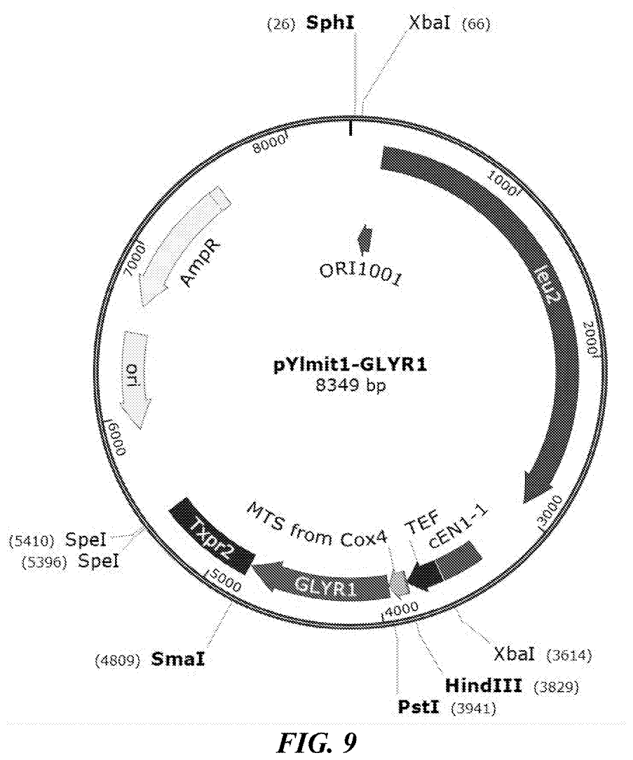

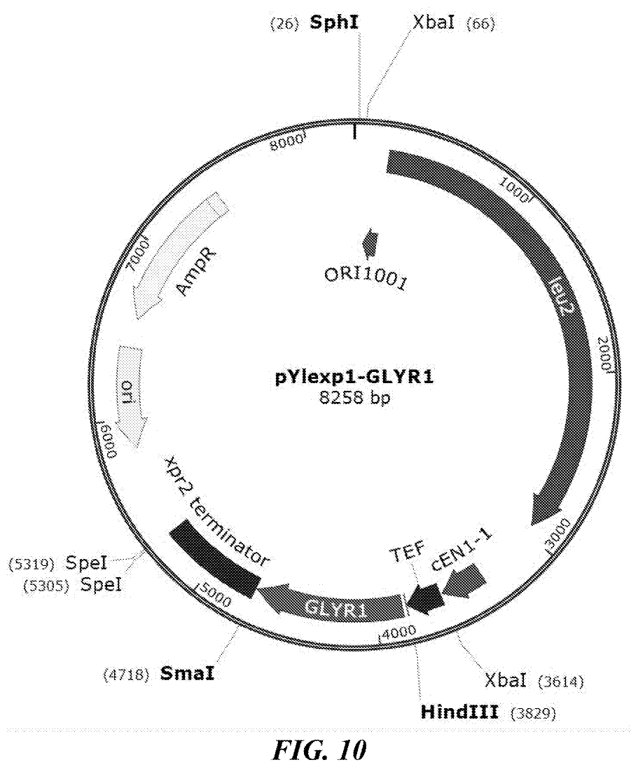

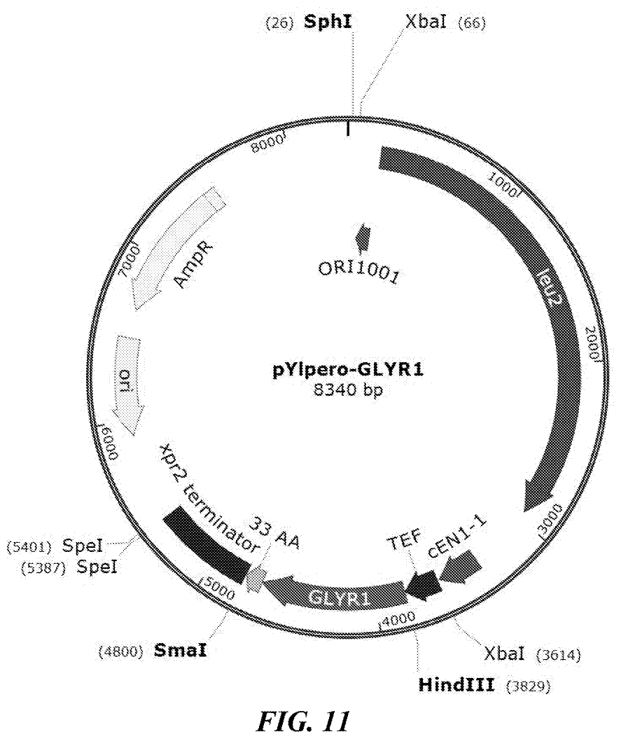

The gene encoding GLYR1 from A. thaliana was expressed in the different organelles by using the developed expression vectors, The vector pYlmit1-GLYR1 was constructed to express GLYR1 in yeast mitochondria by insertion of GLYR1 gene into plasmid pYlmiti of the cleavage sites of Pstl and Smal (FIG. 9). in expression vector pYlpero-GLYIR1 C-terminal 33-amino acid from ICU1 containing peroxisomal targeting signal (PTS) type (PTS1) signal enables the expressed GLYR1 to localize in yeast peroxisome (FIG. 11). The expression vector pYlexp1-GLYR1 was developed to express GLYR1 without any signal peptides, and gene product was retained in yeast cytosol (FIG. 10). The expression GLYR1 cassettes from pYlmit1-GLYR1, pYlpero-GLYR1 and pYlexp1-GIXR1 were inserted into ptiR.A3loxp, and then integrated into the genome of Y. lipolpica GLO9 by yeast transformation. Accordingly, the new strains Y. lipolytica GLO10 expressing GLYR1 from A. thaliana in mitochondria, GLO11 expressing GLYR1 in peroxisome and GLO12 expressing GLYR1 in cytosol were constructed for glycolic acid production.

### Example 3

### Expression of Additional Genes to Improve Glycolic Acid Production

The 1.30-kb DNA fragment of ace4 encoding isocitrate lyase (ecj JW3975) from E. coil was amplified by PCR with primers EcAceA-F1 (SEQ ID NO 20) and. EcAceA-R1 (SEQ ID NO 21) by using genome DNA of E. coil K12 MG1655. The sequences of EcAceA-F1 and EcAceA-R1 are listed below.

EcAceAF1:
GGCGCACTGCAGATGAAAACCCGTACACAACAAA
EcAceAR1:
GCAATTCCCGGGTTAGAACTGCGATTCTTCAGTGGA

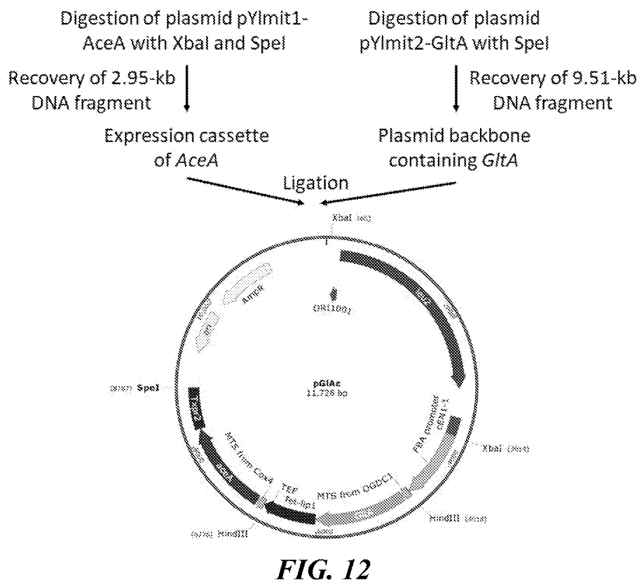

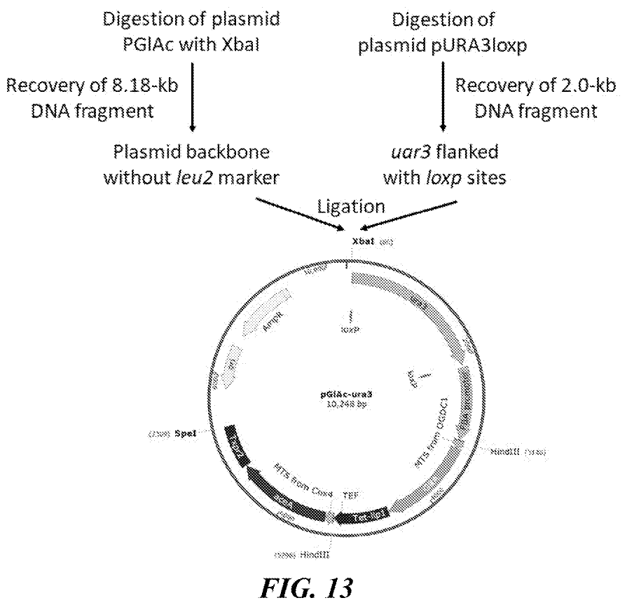

The PCR product was digested with PstI and SmaI, and inserted into the digested plasmid pYlmit1 to generate pYlmit1-AceA. In plasmid pYlmit1-AceA, expression of AceA was fused with signal peptide of Cox4, so AceA. could be translocated into yeast mitochondria. Similarly, pYlmit2-G1tA was constructed to express gliA encoding citrate synthase (ecj:JW0710) from E. coli, and the expressed enzyme was present in mitochondria because of the signal peptide from OGDC used for targeting to cellular compartment. The plasmid pYlmitl-AceA was digested Xbal and SpeI, and then 2.95-kb DNA fragment containing expression cassette of AceA was recovered (FIG. 12), The recovered 2.95-kb DNA fragment was inserted into Spe1 restriction site of plasmid pYlmit2-GltA. The new plasmid pGlAc contained expression cassettes of both A.ceA. and GltA (FIG. 12). The plasmid pGlAc was digested with Xbal to remove leu2 marker and DNA fragments responsible for replication in Y lipolytica, and 2.0-kb DNA fragment of uar3 flanked with loxp sites from plasmid pURA3loxp was inserted into Xhal site of pGlAc (FIG. 13), The new plasmid was designated pGlAc-ura3 (FIG. 13). The linearized plasmid pGlAc-ura3 was integrated into Y. lipolytica expressing GLYR1 from A. thaliana in mitochondria. The new strain was specified as Y. lipolytica GLO16.

Malate dehydrogenase (MDH) from Streptomyces coelicolor A3(2) was engineered to alter its co-factor preference with NADP⁺ instead of NAD⁺. The gene mut-MDII was synthesized with codon optimization of Y. lipolytica (SEQ ID NO 22), and mut-MDH was cloned by using mitochondrial expression vector pYlmit1. Expression of cassette of/mut-MDH was integrated into Y. lipolytica expressing GLYR.1 from A. thaliana in mitochondria to form the strain GLO20. The strains including GLO10, GLO16 and GLO020 were used for production of glycolic acid.

### Example 4

### Production of Glycolic Acid From Glucose and Acetic Acid By Y. Lipolytica

The culture media was composed of 2.5 g/L peptone, 6.7 g/L YNB without amino acids, and acetic acid or glucose as carbon source. For the media containing acetic acid, pH of the media was adjusted to 7.0 by using NaOH. The cultivation for production of glycolic acid was implemented in 250-mL flask containing 50 ml culture media, at 28° C. and 200 rpm without pH control.

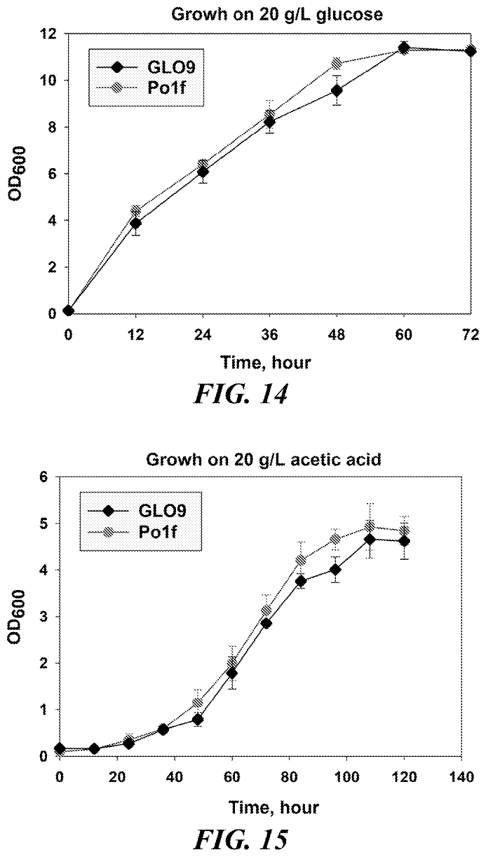

By measurement of absorbance at 600 nm (OD₆₀₀) of the culture every 12 hours, the growth of GLO9 and the control strain, Polf was quantified (FIG. 14, FIG. 15). There was no obvious deficient growth observed for strain GLO9 with disrupted genes of ms1 and ms2 encoding malate synthase on both glucose and acetic acid (FIG. 14, FIG. 15). The strains grown on 20 g/L, of glucose exhibited higher growth rates and higher final biomass yield than those of the strains grown on 20 g/L acetic acid.

To test the strains for glycolic acid production, samples of the culture were collected for measurement of glycolic acid. One mL culture was centrifuged at 13000 rpm, and the supernatant was used for determination of residual glucose or acetic acid in the medium and produced glycolic acid. The concentration of glucose, acetic acid and glycolic acid was quantified by using the external standard method with high-performance liquid chromatography (HPLC).

As shown in FIG. 16 and FIG. 17, the strains GLO10, GLO11 and GLO12 bearing the gene encoding GLYR1 from A. thaliana could produce glycolic acid from both glucose and acetic acid, whereas strain GLO9 without GLYR1 could not produce glycolic acid. By using 40 g/L glucose as substrate after 96 h cultivation, the strain GLO10 produced 3.53 g/L glycolic acid, which was higher than both GLO11 (3.37 g/L glycolic acid) and GLO12 (2.08 g/L glycolic acid) (FIG. 16). By using 30 g/L of acetic acid after 96 h cultivation, the strain GLO10 reached the highest glycolic acid content, 5.68 g/L (FIG. 17). The results indicated that expression of GLYR1 in either mitochondria or peroxisome is beneficial for glycolic acid production compared with cytosolic expression of GLYR1. For use of both glucose and glycolic acid, the strain GLO10 expressing mitochondrial GLYR1 reached the higher titer for glycolic acid production than GLO11 and GLO112. Furthermore, acetic acid was a more favorable carbon source for production of glycolic acid than glucose.

Because the strain expressing mitochondrial GLYR1 showed a better performance for glycolic acid production from both glucose and acetic acid, it was further genetically modified to improve glycolic acid production. As shown in FIG. 18, glycolic acid production from 40 g/L, glucose by the strains GLO10, GLO15 and GLO16 were detected. The strain GLO16 expressing the genes of aceA and gltA from E. coli produced 4.29 g/L glycolic acid after 96 h cultivation. Among the strains GLO10, GLO15 and GLO16, GLO16 was most productive for glycolic acid production (FIG. 18). Therefore, glycolic acid produced by strain GLO16, glucose content and cell growth were monitored every 12 hours (FIG. 19).

The strains GLO10, GLO15 and GLO16 were also used for production of glycolic acid by using acetic acid as carbon source (FIG. 20). However, there was no obvious difference observed for their capability for production of glycolic acid (FIG. 20). The strain GLO20 was developed by introducing a mutant gene mut-A1DH encoding a modified malate dehydrogenase (MDH) from S coelicolor A3(2), Glycolic acid production from acetic acid was improved by strain GLO20. The final titer of glycolic acid production at 96 hours reached 6.74 g/L, representing a yield at 0.22 g glycolic acid/g acetic acid.

### Example 5

### Treatment of Food Waste for Production of VFA and Use of Resultant VFA for Production of Glycolic Acid

A novel AD was developed as a part of this disclosure for efficient VFA production from waste through arresting methanogenesis and accelerating acidogenesis. The anaerobic sludge inoculum was obtained from a primary sedimentation tank at the wastewater treatment plant (WWTP) in Pullman, Wash. The sludge was transferred into sterile bottles purged with nitrogen gas to ensure anaerobic conditions, and then stored at 37 □ for one week to minimize the degradation of organic compounds in the sludge.

The food waste was collected from a student cafeteria at Washington State University in Pullman, Wash., USA. The food waste was mixed with rice, noodles, meat, and all kinds of vegetables and fruits. The characteristics of seed sludge and food waste are shown in table 3.

TABLE 3
Characteristics of sludge and food waste

| Parameter | Food waste | Inoculum |
|---|---|---|
| Total Solids (TS) (%) | 28.52 ± 0.3 | 1.52 ± 0.1 |
| Volatile Solids (VS) (%) | 26.66 ± 0.3 | 1.10 ± 0.1 |
| VS/TS (%) | 93.47 ± 0.1 | 72.46 ± 0.1 |
| pH | — | 7.5 |

The VFA production process was conducted in a 7.5-L fermenter (NBS Bioflo-110) with a 5-L working volume. The mixed liquor was designed to contain 15% total solid of 2,500 g food waste and 2,500 g anaerobic sludge. The confine medium was purged with nitrogen for 20 min and capped tightly with butyl rubber to maintain anaerobic conditions. AD process was carried out by control of temperature (60-80° C.), agitation speed at 300 rpm, pH at 7.0, and without aeration. As shown in FIG. 21, more than 50 g/L VFA, mainly consisting of acetic acid, was produced from food waste by this novel AD process.

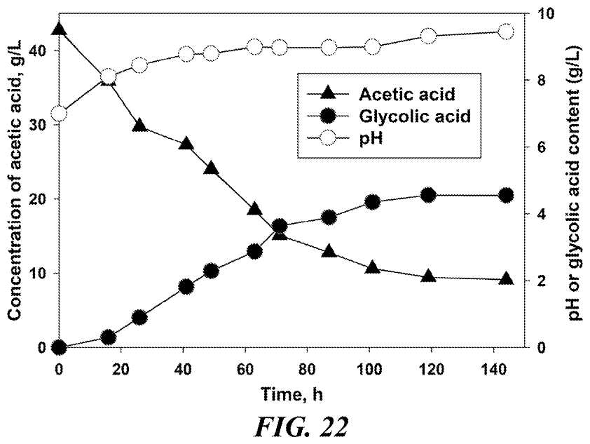

After centrifugation at 13,000 g for 15 minutes, the liquid phase was separated from the product of food waste digestion. The effluent enriched with VFA was used to culture strain GLO20. The media contained around 42 g/L acetic acid generated from food waste, 2.5 g/L peptone and 6.7 g/L YNB without amino acids. As shown in FIG. 22, the strain could produce more than 4.0 g/L glycolic acid in shaking flask at 144 hour, and pH increased from 7.0 to 9.45 during cultivation. The pH change was mainly due to utilization of acetic acid. The production of bio-based glycolic acid from organic waste was achieved by this hybrid process.

### REFERENCES

### Patent Citations

Philippe Soucaille. Glycolic acid production by fermentation from renewable resources. Pub. No.: WO/2007/141316, International Application No.: PCT/EP2007/055625, Publication Date: Dec. 13, 2007, International Filing Date: Jun. 7, 2007

Philippe Soucaille. Method for producing high amount of glycolic acid by fermentation. Pub. No.: WO12010/108909, International Application No.: PCT/EP2010/053758, Publication Date: Sep. 30, 2010, InternationalFiling Date: Mar. 23, 2010

Gregory Stephanopoulos, Zheng-Jun Li, Brian Pereira. Microbial production of renewable glycolate. Pub. No.: US 2017/0121717 A1, Publication Date: Apr. 5, 2017

Outi KOIVISTOINEN, Joosu. KUIVANEN, Peter Richard, Merja Penttild. Eukaryotic cell and method for producing glycolic acid. International Publication Number: WO 2013/050659 A1, International Publication Date: Apr. 11, 2013

### Non-Patent Citations

Agler, M. T., B. A. Wrenn, S. H. Zinder and L. T. Angenent (2011). Waste to bioproduct conversion with undefined mixed cultures: the carboxylate platform. Trends in Biotechnology 29(2): 70-78.

Blazeck, J., L. Liu, H. Redden and H. Alper (2011). Tuning gene expression in Yarrowia hpolytica by a hybrid promoter approach. Applied and Environmental Microbiology 77(22): 7905-7914.

Deng, Y., N. Ma, K. Zhu, Y. Mao, X. Wei and Y. Zhao (2018). Balancing the carbon flux distributions between the TCA cycle and glyoxylate shunt to produce glycolate at high yield and titer in Escherichia coli. Metabolic engineering 46: 28-34.

Ge, Y., P. Song, Z. Cao, P. Wang and G. Zhu (2014). Alteration of coenzyme specificity of malate dehydrogenase from Streptomyces coelicolor A3 (2) by site-directed mutagenesis. J Genet. Mol. Res 13: 5758-5766.

Green Michael R, Sambrook Joseph (2012). Molecular Cloning: Laboratory Manual (Fourth Edition) Cold Spring Harbor Laboratory Press

Li, W., J. Chen, C.-X. Liu, Q.-P. Yuan and Z.-J. Li (2019). Microbial production of glycolate from acetate by metabolically engineered Escherichia coli. J Journal of biotechnology 291: 41-45.

Markham, K. A. and H. S. Alper (2018). Synthetic biology expands the industrial potential of Yarrowia lipolytica. Trends in Biotechnology 36(10): 1085-1095.

Salusjärvi, L, S. Havukainen, O. Koivistoinen and M. Toivari (2019). Biotechnological production of glycolic acid and ethylene glycol: current state and perspectives. Applied Microbiology and Biotechnology 103(6): 2525-2535.

Yamane, T., H. Sakai, K. Nagahama, T. Ogawa, M. Matsuoka (2008). Dissection of centromeric DNA from yeast Yarrowia lipolytica and identification of protein-binding site required for plasmid transmission. Journal of Bioscience and Bioengineering 105(6): 571-578.

1. A system for biosynthesis of glycolic acid, comprising a first expression cassette comprising a polynucleotide encoding glyoxylate reductase operably linked to an expression control sequence and a second expression cassette comprising a polynucleotide encoding a NADP⁺-dependent malate dehydrogenase operably linked to an expression control sequence.

2-4. (canceled)

5. The system of claim 1, wherein the glyoxylate reductase comprises Glyoxylate Reductase 1 (GLYR1).

6. The system of claim 5, wherein the GLYR1 comprises Arabidopsis thaliana GLYR1.

7. The system of claim 5, wherein the GLYR1 comprises SEQ ID NO: 17.

8. (canceled)

9. The system of claim 1, wherein the NADP+-dependent malate dehydrogenase comprises SEQ ID NO: 22.

10. The system of claim 1, wherein the glyoxylate reductase and/or the NADP+-dependent malate dehydrogenase comprises a mitochondria targeting signal and/or a peroxisome targeting signal.

11-12. (canceled)

13. The system of claim 10, wherein the mitochondria targeting signal is a leading sequence from COX4 (YALI0F03567 g) or a leading sequence from OGDC1 (YALI0E33517 g).

14. (canceled)

15. The system of claim 10, wherein the mitochondria targeting signal comprises SEQ ID NO: 19.

16-17. (canceled)

18. The system of claim 10, wherein the peroxisome targeting signal is a 33-amino acid peroxisome targeting signal from isocitrate lyase (ICL1).

19. (canceled)

20. The system of claim 1, wherein the expression control sequence comprises a promoter that is functional in a yeast cell and/or a terminator that is functional in a yeast cell.

21. The system of claim 20, wherein the promoter comprises a Tef promoter.

22. (canceled)

23. The system of claim 20, wherein the terminator comprises xpr2.

24. The system of claim 1, wherein the expression cassette is included in a yeast transformation vector.

25-26. (canceled)

27. The system of claim 1, further comprising a gene cassette comprising a polynucleotide encoding an isocitrate lyase enzyme operably linked to an expression control sequence, a gene cassette comprising a polynucleotide encoding a citrate synthase operably linked to an expression control sequence, or a combination thereof.

28. (canceled)

29. The system of claim 1, further comprising a gene deletion cassette for deletion of a malate synthase gene.

30. The system of claim 1, comprising a gene deletion cassette for deletion of malate synthase 1 (ms1) and a gene deletion cassette for deletion malate synthase 2 (ms2).

31. A recombinant yeast cell comprising a knockout of at least one malate synthase gene selected from malate synthase 1 (ms1) and malate synthase 2 (ms2).

32-40. (canceled)

41. A recombinant yeast cell transformed with the system of claim 1, wherein the recombinant yeast cell produces an increased level of glycolic acid, relative to a control yeast cell.

42-49. (canceled)

50. A method of producing a recombinant yeast cell, the method comprising:
introducing into a yeast cell a system of claim 1 to produce a recombinant yeast cell;
culturing the recombinant yeast cell under conditions sufficient to allow development of a yeast cell culture comprising a plurality of recombinant yeast cells;
screening the recombinant yeast cells for expression of a polypeptide encoded by the system; and
selecting from the yeast cell culture a recombinant yeast cell that expressed the polypeptide.

51-60. (canceled)

61. A method of producing volatile fatty acids (VFAs) from organic waste, the method comprising inoculating a culture medium with an anaerobic sludge and culturing the anaerobic sludge with the organic waste under anaerobic culture conditions sufficient to convert the organic waste into VFAs.

62-66. (canceled)
